(19) United States
(12) Patent Application Publication
(10) Pub. No.: US 2018/0353077 A1
(43) Pub. Date: Dec. 13, 2018

(54) SIGNAL COMPENSATION OPTICAL COHERENCE TOMOGRAPHY SYSTEM AND METHOD

(71) Applicants: National University of Singapore, Singapore (SG); University of French Polynesia, Faa'a (FR); Singapore Health Services Pte Ltd, Singapore (SG)

(72) Inventors: Nicholas Strouthidis, London (GB); Michael Julien Alexandre Girard, Singapore (SG); Jean Martial Mari, Tahiti (FR); Jodhbir Mehta, Singapore (SG); Foin Nicolas, Singapore (SG)

(21) Appl. No.: 15/570,694

(22) PCT Filed: Apr. 29, 2016
(86) PCT No.: PCT/SG2016/050202

§ 371 Date: Oct. 30, 2017

Related U.S. Application Data
(60) Provisional application No. 62/190,424, filed on Jul. 9, 2015.
(60) Provisional application No. 62/154,854, filed on Apr. 30, 2015.

(51) Int. Cl. A61B 5/00; A61B 3/10; A61B 5/02; G01B 9/02

(52) U.S. Cl. CPC A61B 5/0066; A61B 3/102; A61B 5/0084; A61B 2576/00; A61B 5/742; A61B 5/02007; G01B 9/02091; A61B 5/7264

(57) ABSTRACT

An optical coherence tomography system and method. The system comprises a detector for detecting an optical coherence tomography signals from a sample; a processing unit for converting the detected signals to raw data; a compensation unit for performing compensation to generate compensated data; and a display for displaying at least the compensated data.

### FIELD OF INVENTION

The present invention relates broadly to a signal compensation optical coherence tomography system and method.

### BACKGROUND

Optical coherence tomography (OCT; an optical non-invasive imaging modality) imaging has been used increasingly, for example to monitor retinal disorders, and there also has been a growing interest for its use in corneal applications in recent years. Clinically, anterior segment OCT (ASOCT) has shown great promise in a variety of indications, for example, in (1) mapping corneal topography and tomography with high resolution, (2) evaluating corneal wounds, incisions, scars, inflammation or infective lesions, (3) assessing the posterior cornea in terms of topography and structural changes (e.g., diagnosing Descemet's membrane detachment), and in (4) evaluating and monitoring structural changes following corneal transplants and corneal refractive surgeries.

As ASOCT hardware and software continues to improve, there is an increasing use of the ASOCT in daily clinical practice, as well as for ‘real-time’ intraoperative assessments. However, for example corneal images acquired with OCT suffer from visibility artifacts, also observed in for example retinal or optic nerve head images, which are the direct result of light attenuation and sensitivity falloff with depth.

There are several challenges in the application of OCT imaging to different uses, and also different challenges for different applications. For example, because the cornea is prolate in shape, biomechanically heterogeneous, and of varying thickness, ASOCT A-scans of an individual cornea exhibits signal attenuation at varying depths, thus emphasizing the need for ‘local’ image processing (as opposed to ‘global’ image processing).

While the image quality of OCT has been significantly improved to visualize deep tissue ocular structures without the need of surgical interventions, OCT image quality is still greatly hampered by the presence of artifacts and by poor tissue visibility in the deepest layers. This is due to for example signal attenuation, whereby signal strength diminishes as a function of tissue depth. Such phenomena are a barrier to clinical applications and prevent the diagnosis and risk management of multiple ophthalmic pathologies.

Current techniques to enhance OCT image quality in Ophthalmology are:

1) Standard contrast enhancement through pixel-intensity exponentiation (as used in edge detection and segmentation algorithms). However such techniques are very poor/crude as they make artifacts more noticeable and reduce the visibility of deep-tissue layers (Girard et al., IOVS. 2011; 52(10):7738-48). These techniques are also not available in real time.
2) Signal Averaging. This technique performs multiple imaging acquisitions (between 1 and 100 times) of the same tissue plane and produces an ‘average’ pixel intensity image, thus reducing speckle noise. However, signal averaging cannot increase contrast, remove artifacts, and improved deep-tissue layer visibility.
3) Enhanced Depth Imaging (EDI). This is a hardware technique to improve the visibility of deep-tissue layers. It consists in modifying the distance between the tissue of interest and the OCT objective during acquisition. However, EDI cannot remove shadow artifacts and cannot increase contrast (as opposed to compensation). Furthermore, compensation has been demonstrated to be superior to EDI in improving deep-tissue visibility (Girard et al., IOVS. 2015; 56(2):865-74). Finally, applying compensation to EDI images is still possible and results in the best image enhancement possible (Girard et al., IOVS. 2015; 56(2):865-74).

Current OCT systems are also not able to map attenuation-corrected tissue optical-properties.

Embodiments of the present invention provide an optical coherence tomography system and method that seek to address at least one of the above problems.

### SUMMARY

In accordance with a first aspect of the present invention, there is provided an optical coherence tomography system comprising a detector for detecting an optical coherence tomography signals from a sample; a processing unit for converting the detected signals to raw data; a compensation unit for performing compensation to generate compensated data; and a display for displaying at least the compensated data.

In accordance with a second aspect of the present invention, there is provided an optical coherence tomography image processing method comprising detecting raw optical coherence tomography data from an optical coherence tomography imaging system; applying of a compensation step for generating compensated data based on the raw data; and displaying at least the compensated data.

### BRIEF DESCRIPTION OF THE DRAWINGS

Embodiments of the invention will be better understood and readily apparent to one of ordinary skill in the art from the following written description, by way of example only, and in conjunction with the drawings, in which:

FIGS. 1 A, B illustrate better visibility of the posterior lamina cribrosa surface in OCT images of the human optic nerve head, according to an example embodiment.

FIGS. 2 A-H illustrate better visibility of the cornea in OCT images, in particular OCT images of the cornea, along with the maximum penetration depths, compensation curves and A-Scan pixel intensities, according to example embodiments.

FIGS. 3 A, B illustrate exacting regions of interests locations used to calculate the interlayer contrast for compensated OCT images, according to example embodiments.

FIGS. 4 A-H illustrate OCT image contrast, corneal stroma visibility and low noise over-amplification, for identifying the corneal endothelium and corneal thickness of one subject, according to example embodiments.

FIGS. 5 A-H illustrate OCT image contrast, corneal stroma visibility and low noise over-amplification, for identifying the corneal endothelium and corneal thickness of another subject, according to example embodiments.

FIGS. 6 A-H illustrate OCT image contrast, corneal stroma visibility and low noise over-amplification, for identifying the corneal endothelium and corneal thickness of another subject, according to example embodiments.

FIGS. 7 A-H illustrate OCT image contrast, corneal stroma visibility and low noise over-amplification, for identifying the corneal endothelium and corneal thickness of another subject, according to example embodiments.

FIGS. 8 A-F illustrate OCT images of the cornea, along with the maximum penetration depths, compensation curves and A-Scan pixel intensities, according to example embodiments.

FIG. 9 shows a table of inter-layer contrasts (across the corneal endothelium) at different vertical locations for four subjects, according to example embodiments.

FIG. 10 shows the posterior lamina cribrosa surface in an OCT image of the human optic nerve head with different TE values indicated as horizontal lines for AC compensation in a real time workflow embodiment.

FIG. 11 shows the functional ƒ to be minimized with respective values for different TE values tested for the image in FIG. 10, according to a real time workflow embodiment.

FIG. 12 shows the resultant compensated image for the optical TE value, with the compensation limit indicated as a horizontal line, for real time display, according to a real time workflow embodiment.

FIGS. 13A-D illustrate application of compensation techniques according to example embodiments to cardiovascular OCT images of the coronary artery.

FIG. 14 illustrates compensation performed on baseline OCT images and compared with matched histological images, according to example embodiments.

FIG. 15 shows a schematic diagram illustrating an OCT system and associated data and command flows according to an example embodiment.

FIG. 16 shows a flow chart illustrating an optical coherence tomography image processing method according to an example embodiment.

### DETAILED DESCRIPTION

Embodiments of the present invention aim to combine OCT hardware and compensation software to achieve either real-time or deferred compensation in ophthalmology. The compensation applied in different embodiments may include one or more of:
Standard Compensation (Girard et al., IOVS. 2011; 52(10):7738-48).

Standard Compensation (SC) is a post-processing technique, now recognized by the present inventors to be suitable for incorporation into an OCT system for deferred or real time compensation, that can be applied to OCT images under the assumptions that light attenuation and backscattered light are proportional, and that A-Scan (a single OCT acquisition line, so that OCT images are formed of multiple lines) signals are fully attenuated at infinite depth. Using SC, a compensated image can be produced through pixel intensity amplification, theoretically without depth limitation. Such amplification aims to enhance ‘weak signals’ and is progressively increased with depth using Equation 1-a to produce the compensated intensities I_{i,j}^{SC}. In Equation (1) below, M_{i,j} is the compensation profile (1/M_{i,j} being the amplification factor) for each A-scan j and A-scan pixel at depth i (i=0: top of the image; i=N: bottom of the image); n is an exponent that controls contrast (typically 1 or 2).

$$\{ \begin{matrix}{I}_{i , j}^{SC} = \frac{{I}_{i , j}^{n}}{2. {M}_{i , j}} & ( a ) \\ {M}_{i , j} = {\sum }_{k = i}^{N} {I}_{k , j}^{n} & ( b )\end{matrix} \qquad Equation 1$$

Adaptive Compensation (Mari et al., IOVS, 2013; 54(3):2238-47).

Adaptive compensation (AC) is a post-processing technique, now recognized by the present inventors to be suitable for incorporation into an OCT system for deferred or real time compensation, that can remove noise over-amplification at high depth and is expressed in Equation 2. AC first compares all A-scans and estimates the maximum penetration depth i^{stop} (Equation 2-c) using the global maximum energy profile of all A-scans (Equation 2-b). Then AC computes the standard compensation profile I_{i,j}^{AC} for each A-scan down to the estimated depth i^{stop} (Equation 2-d and -e), and then maintains the compensation coefficient M_{i,j} for each A-scan.

$$\{ \begin{matrix}{E}_{i , j} = {\sum }_{k = i}^{N} {[ {I}_{k , j}^{n} ]}^{2} & ( a ) \\ {E}_{l}^{max} = {max}_{j} {E}_{i , j} & ( b ) \\ {i}^{stop} = 1 \text{/} {E}_{l}^{max} < {10}^{- TE} . {E}_{0}^{max} & ( c ) \\ {M}_{i , j} = \{ \begin{matrix}{\sum }_{k = i}^{N} {I}_{k , j}^{n} & o < {i}_{stop} \\ {\sum }_{k = {i}_{stop}}^{N} & {I}_{k , j}^{n} i \geq {i}_{stop}\end{matrix} & ( d ) \\ {I}_{i , j}^{AC} = {I}_{i , j}^{n} \text{/} 2. {M}_{i , j} & ( e )\end{matrix} \qquad Equation 2$$

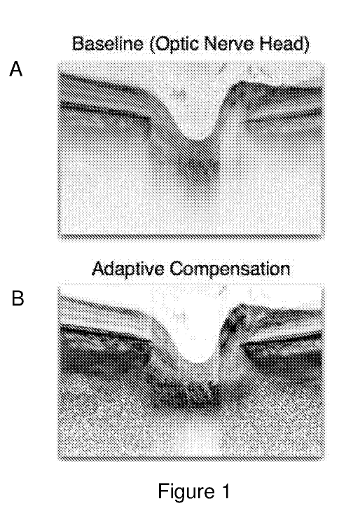

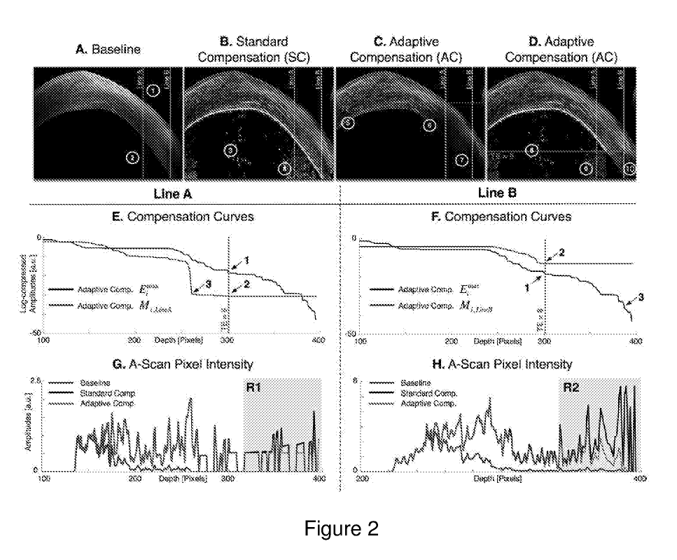

The energy threshold exponent or TE (that controls i^{stop}) can be readily chosen, as only a very small part of the energy remains at the bottom of shallow tissue images. For such images, noise over-amplification can be efficiently removed (providing, for example, better visibility of the posterior lamina cribrosa surface in OCT images of the human optic nerve head; see FIG. 1; or the cornea, see FIG. 2).

Examples of global energy and compensation profiles are shown in FIGS. 2E and 2F for the two A-scans (lines A and B) in FIGS. 2A through 2D. The compensation coefficients M_{i,j} are maintained constant for i greater than i^{stop} using TE=8 (FIGS. 2E, F, arrow 2). It is noted that i^{stop} is the maximum penetration depth and corresponds to the point where only 10^{-TE}=10⁻⁸ of the initial value E^{max}₀ of the global energy profile E^{max}ᵢ remains (FIGS. 2E, F, arrow 1). Maintaining the compensation coefficients constant past a given limit advantageously results in a regularization of the deeper part of the signals, which can be observed in FIGS. 2G and 2H, where the two A-scan signals (baseline, SC, and AC) are plotted as a function of depth. In FIG. 2G (region R1) the baseline signal is essentially background noise. For i greater than i^{stop}=301, the AC signal is not amplified anymore thus resulting in less noise over-amplification than with SC. Although stopping AC signal amplification at arrow 3 in FIG. 2E would be desirable as this corresponds to the corneal endothelium, stopping further at arrow 2 preferably still ensures some level of noise over-amplification control for AC as opposed to SC.

Depth Adaptive Compensation.

In many imaging situations (e.g. OCT imaging of the cornea), biological tissues can be present in both shallow and deep regions of the image. For such images, SC would typically still over-amplify signal at high depth, but AC would stop amplifying beyond a global limit i^{stop} as illustrated in FIGS. 2F and 2H. In FIG. 2F (TE=8), the A-scan would ideally require further amplification down to the end of the line (FIG. 2F, arrow 3 corresponding to the corneal endothelium), but AC maintains M_{i,j} constant prematurely (at arrow 2). Choosing a larger TE value (a smaller threshold) may not solve the problem, as the A-scans containing only noise in the deep regions would be amplified as well. The early maintenance of the M_{i,j} values translates in FIG. 2H to the under-amplification of the AC signal (region R2).

Therefore, it has been recognized by the inventors that the presence of variation in tissue depth in the OCT image is advantageously addressed by the estimation of a compensation limit which is dynamically adapted to the local energy profile, by estimating a maximum penetration depth iⱼ^{stop} for each A-scan j. To that end, each energy line E_{i,j} (Equation 3-a) is preferably individually thresholded (Equation 3-b). It is note that the use of very small respective threshold values (as with the one threshold value in AC) preferably ensures deep tissue signal compensation while preventing noise over-amplification.

$$\{ \begin{matrix}{E}_{i , j} = {\sum }_{k = i}^{N} {[ {I}_{k , j}^{n} ]}^{2} & ( a ) \\ {i}_{j}^{stop} = i \text{/} {E}_{i , j} < {10}^{- TE} . {E}_{0 , j} & ( b ) \\ {M}_{ij} = \{ \begin{matrix}{\sum }_{k = i}^{N} {I}_{k , j}^{n} & i < {i}_{j}^{stop} \\ {\sum }_{k = {i}_{j}^{stop}}^{N} & i \geq {i}_{j}^{stop}\end{matrix} & ( c ) \\ {I}_{i , j}^{DAC} = {I}_{i , j}^{n} \text{/} 2. {M}_{i , j} & ( d )\end{matrix} \qquad Equation 3$$

The depth adaptive compensation (DAC) technique in a preferred embodiment is fully described by Equation 3. Once individual A-scan maximum penetration depths iⱼ^{stop} have been computed, the same regularization of the compensation profile M_{i,j}, as in AC, is performed (Equation 3-c) to generate a DAC image I_{i,j}^{DAC}.

It is noted that to obtain compensated intensities, the compensation equations are preferably discretized, for example with a spatial sampling Δ equal to 1. When the compensated intensities are divided by the physical value of Δ, they preferably represent a quantitative measure of light attenuation.

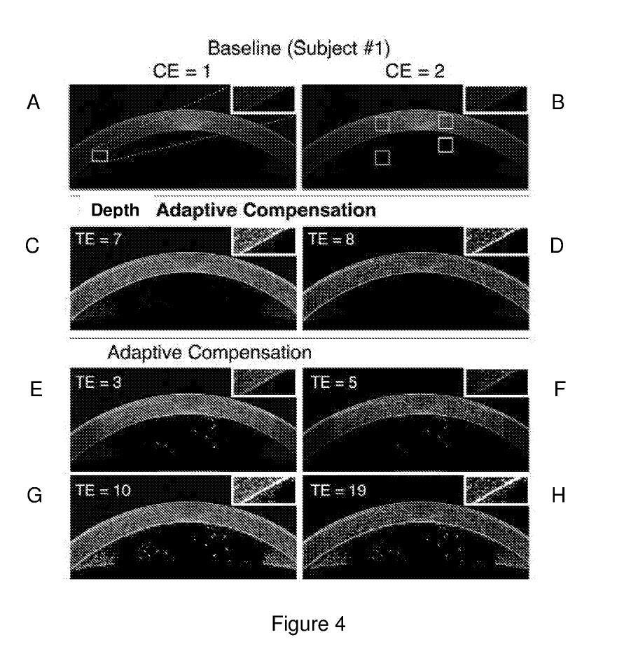

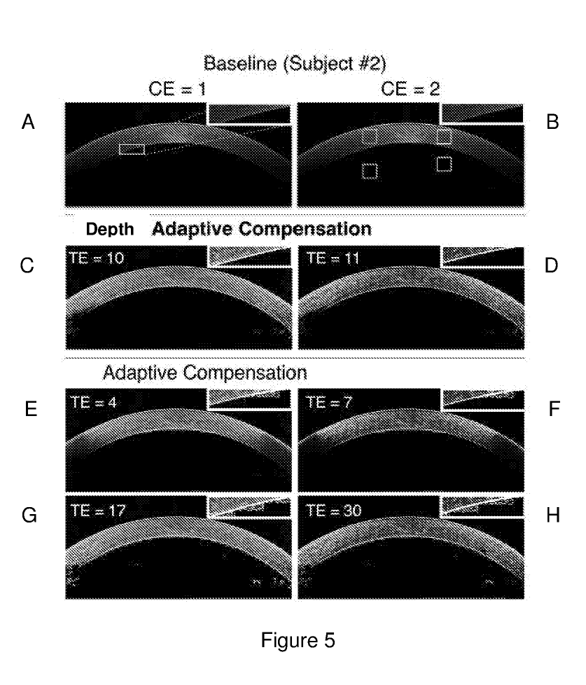

Fourier-domain ASOCT images of the cornea (horizontal B-scan; ×20 signal averaging) of the right eye of four human subjects (2 healthy eyes and 2 eyes exhibiting corneal scars) are shown in FIGS. 4-7. In preferred embodiments, compensation (SC, AC, or DAC) is applied to the raw format, such as IRaw for a RTVue (Optovue, Inc., Fremont, Calif.). of each ASOCT image in real-time.

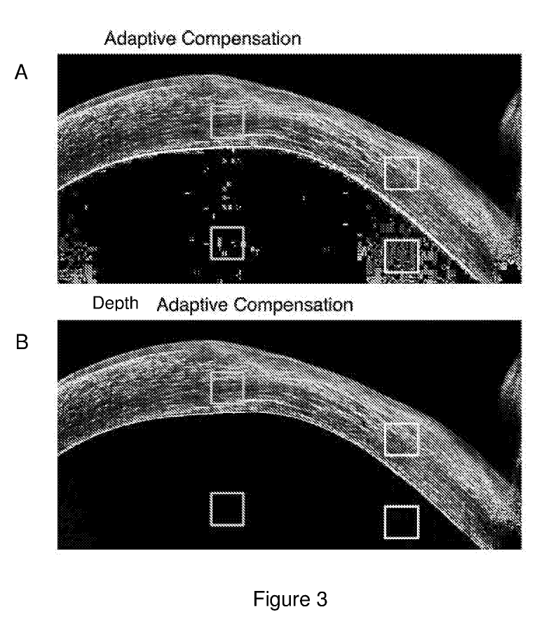

For test-bedding purposes, one may first qualitatively assess the performance of the different compensation algorithms, for example DAC and AC, for various TEs when pertinent and with contrast exponents (CEs, noted as n in the equations (1), (2) and (3), the exponent which may be applied to the OCT signal before or after compensation) of 1 (no contrast enhancement) or 2 (high contrast enhancement). The performance of DAC was also assessed quantitatively by computing the interlayer contrast defined as:

$$inter \text{-} layer contrast = \vert \frac{{I}_{3} - {I}_{4}}{{I}_{3} + {I}_{4}} \vert \qquad ( 4 )$$

where I₃ is the mean pixel intensity of a region of interest (ROI) located within the corneal stroma, and I₄ is that within a deeper region of the anterior chamber (i.e., background). In cornea OCT images, the inter-layer contrast is a measure of corneal endothelium visibility that varies between 0 (poor visibility) and 1 (high visibility). It was calculated at two different locations (central and peripheral) that are typically over-amplified by SC and AC (compare FIG. 3a for AC). The ROIs, indicated as boxes in FIGS. 3a,b, were 60×60 pixels in size for images. Finally, for a given subject, the exact same ROI locations were used to calculate the interlayer contrast for the SC, AC, and DAC images (compare FIGS. 3a,b for AC and DAC respectively).

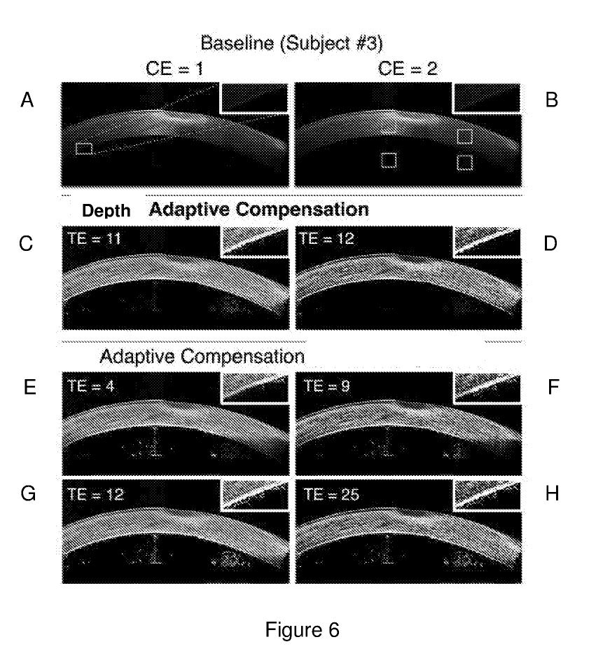

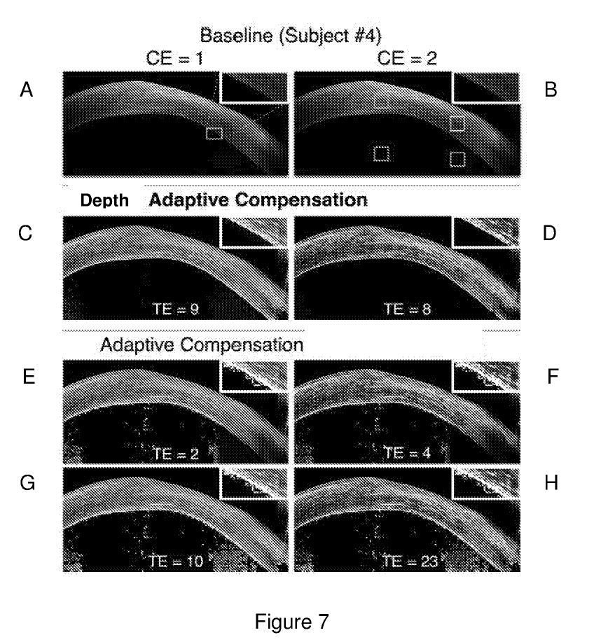

All baseline corneal ASOCT images (the initial images provided by the existing OCT imaging devices) exhibited non-uniform illuminations with especially poor tissue visibility at the cornea periphery (FIGS. 4-7a, b). Additionally, baseline images from unhealthy subjects exhibited shadowing of the corneal stroma due to scarring (FIGS. 6-7a,b). For all images, AC with low TE values restored corneal stroma homogeneity in the central region but not at the cornea periphery where the signal remained poor (FIGS. 4-7;e,f). AC with high TE values provided excellent homogeneity of the corneal stroma in all subjects (FIGS. 4-7;g,h), but at the cost of strong and unwanted noise over-amplification in the deepest corneal layers. Furthermore, noise over-amplification was present in all images (FIGS. 4-7;e-h), which considerably affected corneal endothelium visibility. These results were true for both CE values that were tested (1, 2).

DAC provided considerably superior image contrast than AC did with excellent corneal stroma visibility and low noise over-amplification, thus making corneal endothelium and corneal thickness easily identifiable (FIGS. 4-7;c,d). Further, DAC with CE=2 advantageously allowed contrast enhancement without providing additional noise.

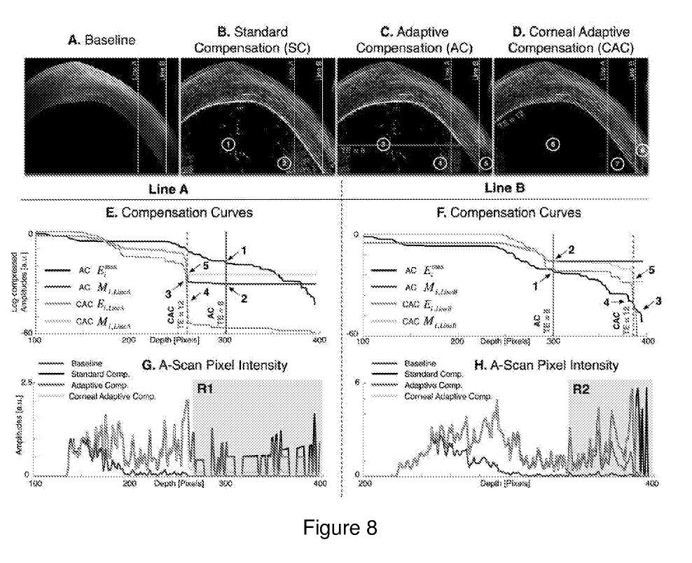

A DAC image of the cornea is shown in FIG. 8D, along with the maximum penetration depths i^{stop}ⱼ for each A-scan (represented as a dashed line that matches the corneal endothelium boundary, for TE=12). For AC, the adaptive maximum penetration depth i^{stop} is plotted as a horizontal line (for TE=8 (FIG. 8C). DAC does not produce signal over-amplification (FIG. 8D, regions 6, 7) as observed with SC (FIG. 8B, regions 1, 2) and with AC (FIG. 8C, regions 3, 4), nor signal under-amplification (FIG. 8D, region 8) as observed with AC (FIG. 8C, region 5).

Compensation profiles are compared between the different techniques (SC, AC, and DAC) for 2 A-scans in FIGS. 8E and 8F. For both shallow and deep tissue A-scans, the DAC limit i^{stop}ⱼ (arrow 5) is consistently close to the ‘ideal’ compensation limit (arrow 3) represented by the corneal endothelium, while the AC limit i^{stop} (TE=8) remains the same (arrow 2). Improved DAC performance is further illustrated in FIGS. 8G and 8H where baseline, SC, AC, and DAC intensity signals are plotted for 2 A-scans. In region R1, DAC efficiently stopped the amplification below the corneal endothelium, while in region R2, the compensation was carried out until the remaining energy was so small that the amplification was stopped, thus preventing saturation.

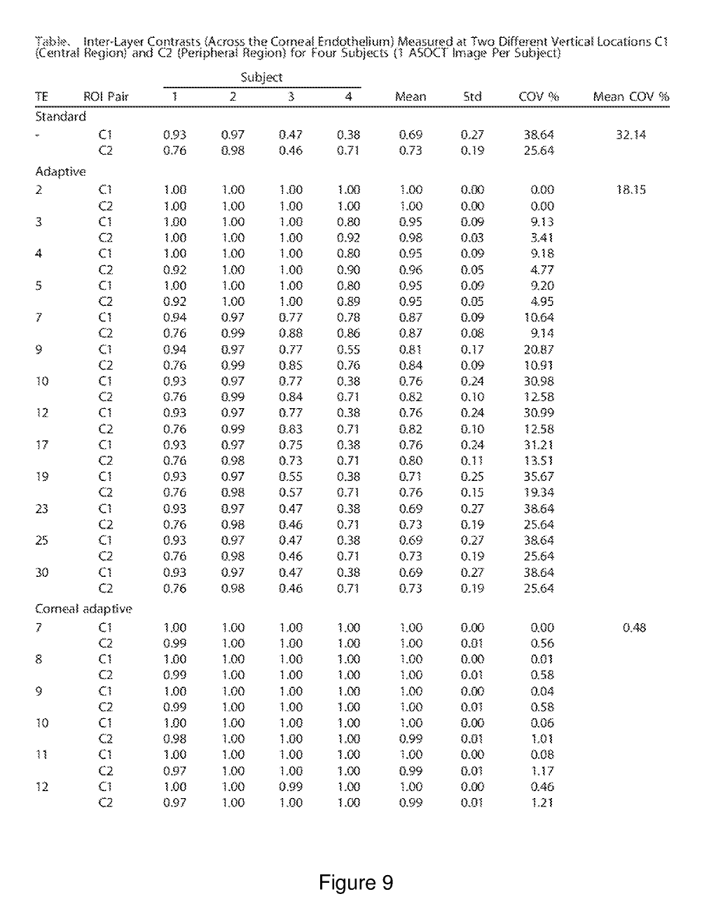

The inter-layer contrasts C1 and C2 obtained respectively from the region pairs illustrated in FIG. 3a,b for each compensation algorithm are provided in the Table shown as FIG. 9 (for SC, AC with 2≤TE≤30, and DAC with 7≤TE≤12) for all four subjects. SC and AC yielded inter-layer contrasts ranging from 0.38 to 1.00, with mean coefficients of variation of 18.15% for AC, and 32.14% for SC. DAC performed even better, with inter-layer contrasts all exceeding 0.97 for a wide range of TE values (between 7 and 12), and with a low mean coefficient of variation (0.48%), suggesting low noise and high corneal stromal endothelium visibility. As mentioned above, DAC was advantageously not affected by signal under-amplification (as observed with AC with low TE) suggesting that compensation is properly applied to all corneal regions. DAC with CE=2 in one embodiment was advantageously able to provide improved contrast without generating additional noise. Such an embodiment advantageously offers clinicians improved detection of heterogeneous corneal features such as may be encountered in cases with corneal scars.

It is also interesting to note that, with DAC, the adaptive limit i^{stop}ⱼ (the boundary when compensation is stopped in Equation 3b) coincides with the corneal endothelium boundary. This is can represent an ideal situation as no further signal amplification should be required past the corneal endothelium. Therefore, DAC may advantageously also be used to automatically segment the corneal endothelium, an important advantage for morphometric or biomechanical characterization of the cornea in vivo. It should be emphasized that corneal endothelium edge detection is advantageously a direct outcome of DAC, and no prior segmentation is actually required for DAC to operate thus advantageously facilitating real time compensation implementation.

The real time workflow in preferred embodiments allows a self-parameterization of the acquisition through a feedback loop where the acquisition parameters and compensation exponent's values can be adjusted before providing the user with the compensated results. In one such embodiment, multiple compensations are performed on a sample acquisition to extract a functional which depends on the depth of the compensation limit and on the values of the regions before and after the limit to determine the optimum threshold exponent TEₒₚₜ.

For embodiments using the AC, the general expression for the search of TEₒₚₜ is:

ƒ:(I^{stop},I_{i,j}^{SC},I_{i,j}^{AC},TE)→ƒ(I^{stop},I_{i,j}^{SC},I_{i,j}^{AC},TE) (5)

TEₒₚₜ=_{TE}^{min}ƒ(I^{stop},I_{i,j}^{SC},I_{i,j}^{AC},TE) (6)

For embodiments using DAC, the general expression for the search is:

ƒ:(I_{I}^{stop},I_{i,j}^{SC},I_{i,j}^{DAC},TE)→ƒ(Iⱼ^{stop},I_{i,j}^{SC},I_{i,j}^{DAC},TE) (7)

TEₒₚₜ=_{TE}^{min}ƒ(Iⱼ^{stop},I_{i,j}^{SC},I_{i,j}^{DAC},TE) (8)

The search may also depend on other parameters such as the CE n, or intermediate compensation values, or the acquisition parameters of the OCT acquisition device. The search for a minimum could also be inverted in the search of a maximum value (max) and the corresponding parameters values.

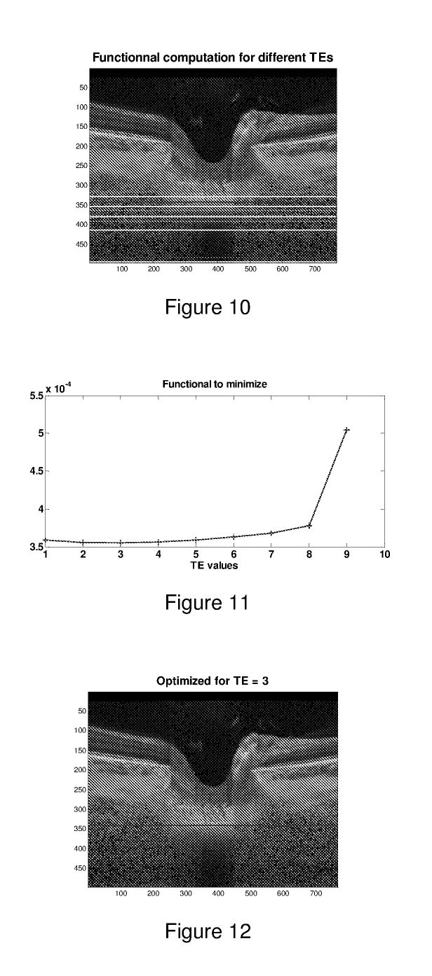

FIG. 10 shows the posterior lamina cribrosa surface in an OCT image of the human optic nerve head with different TE values indicated as horizontal lines for AC compensation in a real time workflow embodiment. FIG. 11 shows the functional ƒ to be minimized with respective values for different TE values tested for the image in FIG. 10, and FIG. 12 shows the resultant compensated image for the optical TE value, with the compensation limit indicated as a horizontal line, for real time display.

The real time workflow in preferred embodiments may further comprise the feedback loop being configured to select an optimum compensation algorithm, such as by performing the above described searches based on the functional of the respective compensation algorithms (Equations (7)-(10)) so select between the AC and the DAC algorithms in one embodiment.

Embodiments of the present invention have a number of applications. To date, corneal diseases are still one of the most common causes of vision loss and irreversible blindness. It is estimated that more than 180 million people worldwide suffer from visual impairment from corneal disease, and over 10 million patients with blindness from ocular surface scarring. Corneal transplantation still remains the main method for restoring vision, once corneal clarity is affected. Recent developments in surgical techniques have enabled surgeons to perform selective replacement of the diseased layer of the cornea, which has led to improved corneal transplant survival and surgical outcomes. Thus, the demand for high-resolution imaging, preferably with real-time compensation according to example embodiments of the present invention, to consistently identify corneal layers, and objective quantification of corneal pathology has increased exponentially. Imaging and objective identification of each diseased layer of the cornea will enable surgeons to plan targeted replacement of that corneal tissue. In addition to assessing corneal and anterior segment parameters in healthy population subjects, example embodiments of the present invention can also provide improved visualization of corneal structure, lesions, and corneal measurements even in patients with corneal edema, infection, or scarring. Such information would be useful to evaluate corneal structure and/or lesions, monitor remodeling following Descemet stripping endothelial keratoplasty, and to guide surgical procedures such as the choice of corneal transplantation, or even type refractive surgery. Example embodiments of the present invention may also find applicability in OCT images of the iris, lens, limbus, trabecular meshwork, and Schlemm's canal. Example embodiments of the present invention can enlarge the panel of techniques available for restoring OCT images from different regions of the eye, which could contribute to improved diagnosis and facilitate automated analysis of ocular biomechanics.

Furthermore, embodiments of the present invention are not limited to ophthalmological applications, but may be applied in other fields such as in Cardiology.

In a particular embodiment, a combination of OCT imaging hardware with a compensation step in the image processing software is provided to achieve compensation of OCT images in cardiology. Current cardiovascular OCT systems such as the ILUMIEN OPTIS PCI Optimization System (St Jude Medical, US) or the Lunawave OFDI Intravascular Imaging System (Terumo corporation, Japan) are limited by the rapid attenuation of the OCT signal into tissue limiting field of view into the arterial wall to only 1 to 2 mm.

Real-time (or deferred) compensation for intravascular cardiovascular OCT imaging may be advantageous for one or more of the following items: 1) Improve the detection of vascular wall tissue with catheter based OCT imaging system; 2) Improve the diagnosis of cardiovascular pathologies (better tissue visibility); 3) Improve the detection of atherosclerotic plaques features, in particular with thin-cap lipid rich plaque at risk of rupture: “vulnerable plaque”; 4) Improve visibility of OCT image during OCT guided Percutaneous Interventions; 5) Addressing one of the main limitations of intravascular OCT: limited penetration due to signal attenuation. Visibility of deeper tissue is lower with OCT as compared with Intravascular Ultrasound (IVUS); 6) Reducing shadowing artefacts which can lead to misinterpretation about plaque morphology; 7) Limit complications that could arise from misinterpretation of OCT images (more accurate diagnosis);

Real-time (or deferred) compensation can therefore increase the utility of OCT in guiding treatment of vascular diseases and contribute to grow the applications of intravascular OCT in interventional procedures, particularly Percutaneous Coronary Interventions.

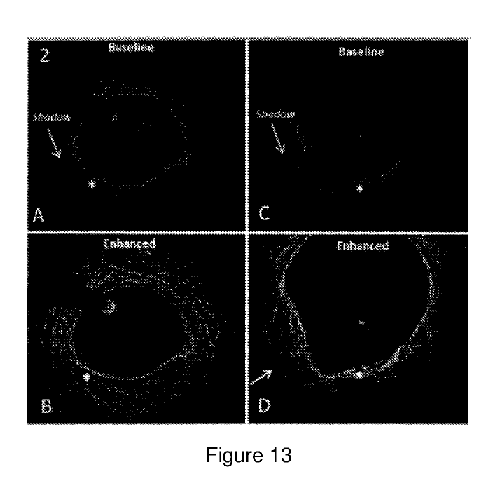

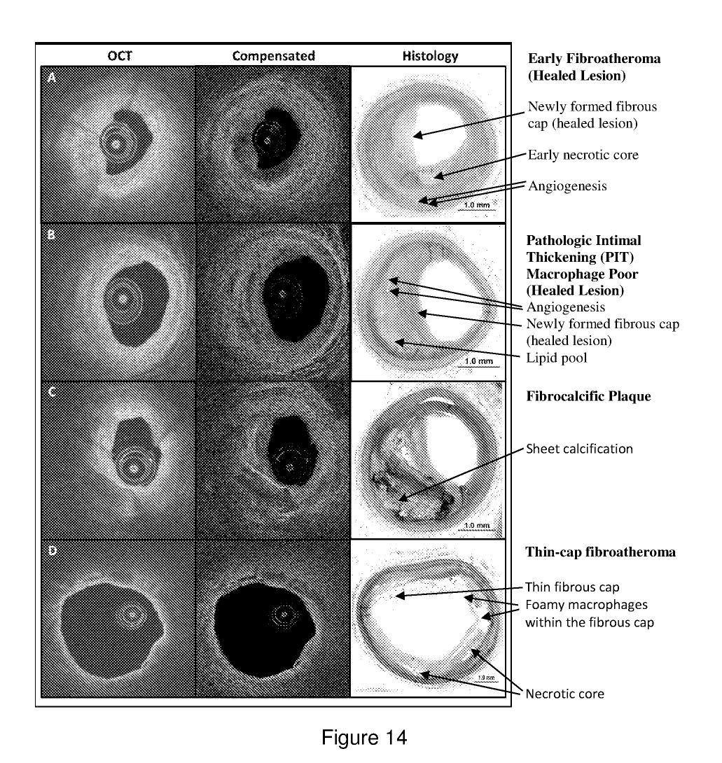

FIGS. 13A-D show application of compensation techniques according to example embodiments to cardiovascular OCT images of the coronary artery. Specifically, improved tissue visibility revealed details within the superficial (FIGS. 13A-D, SC, Exponent n=4,) and outer contour of the athero-sclerotic plaques (arrow, FIG. 13D) that may be critical for interpretation of plaque composition. Such plaques clog blood vessels and their presence can result in heart attack and sudden cardiac death. Visualization of the external vessel contour (External Elastic Laminae) is another critical parameter for assessment of arterial plaque burden. Current intravascular OCT systems often fail to show clearly the External laminae due to OCT signal attenuation in the diseased arterial wall. Compensation techniques was shown to facilitate the detection of such External Elastic Laminae. FIG. 14 shows compensation (middle column) performed on baseline OCT images (left column) and compared with matched histological images (right column) showing the following phenomena: Top row: Early Fibroatheroma (Healed Lesion); second row: Pathologic Intimal Thickening (PIT) Macrophage Poor (Healed Lesion); Third row: Fibrocalcific Plaque; and bottom row: This-cap fibroatheroma. Differing structures are indicated on the histologic images for clarity and it can be observed that such structures are more clearly distinguished in OCT after compensation (SC, Exponent n=4). Enhanced real-time compensated OCT image may be used and advantageous to 1) Improve the diagnosis of cardiovascular pathologies 2) Enhance the assessment of different atherosclerotic plaque morphology 3) Contribute to improve patient outcome and increase usage of catheter based OCT imaging devices to optimize treatment, in particular for coronary and peripheral arterial diseases.

The present specification also specifically discloses apparatus for implementing or performing the operations of the methods. Such apparatus may be specially constructed for the required purposes, or may comprise a device selectively activated or reconfigured by a computer program stored in the device. Furthermore, one or more of the steps of the computer program may be performed in parallel rather than sequentially. Such a computer program may be stored on any computer readable medium. The computer readable medium may include storage devices such as magnetic or optical disks, memory chips, or other storage devices suitable for interfacing with a device. The computer readable medium may also include a hard-wired medium such as exemplified in the Internet system, or wireless medium such as exemplified in the GSM mobile telephone system. The computer program when loaded and executed on the device effectively results in an apparatus that implements the steps of the method.

The invention may also be implemented as hardware modules. More particular, in the hardware sense, a module is a functional hardware unit designed for use with other components or modules. For example, a module may be implemented using discrete electronic components, or it can form a portion of an entire electronic circuit such as an Application Specific Integrated Circuit (ASIC). Numerous other possibilities exist. Those skilled in the art will appreciate that the system can also be implemented as a combination of hardware and software modules.

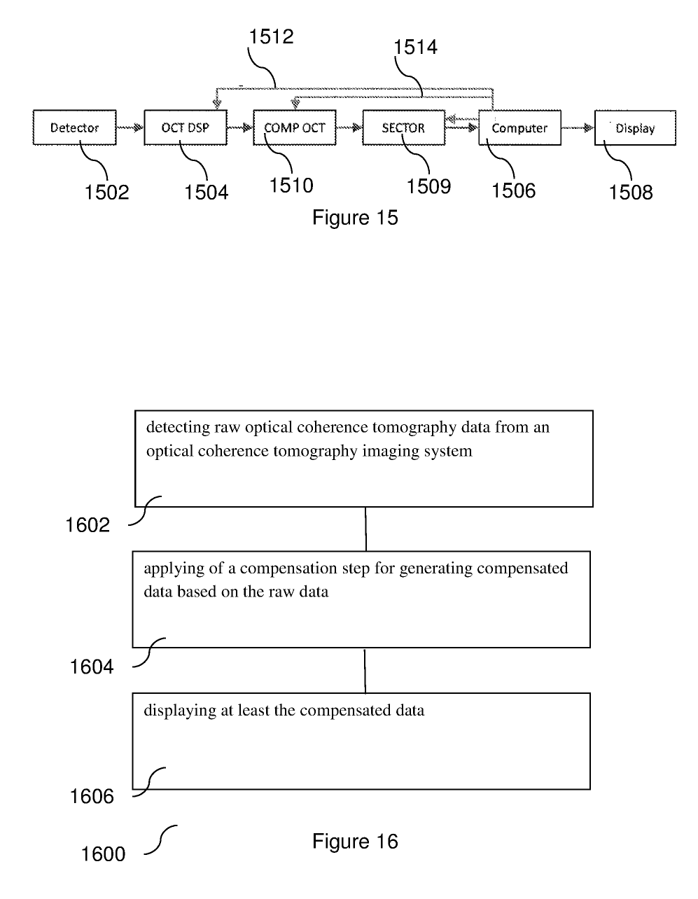

The compensation methods in example embodiment are advantageously fully compatible with the current OCT technology. FIG. 15 shows a schematic diagram illustrating an OCT system 1500 and associated data and command flows. the OCT system comprises a signal detector (1502), which can e.g. be either be a single or an array photodetector, a OCT Digital Signal Processor (DSP) for fast data acquisition and processing (1504), and a computer (1506) and a display device (1508) to allow the user to adjust the settings and visualize the image in real time. Optionally, a polar transform unit (SECTOR) 1509, which may be performed but not necessarily on the user computer 1506, can be provided. Advantageously, the compensation algorithms in the example embodiments described are computationally of low complexity and may be yet further optimized to enable running in real time on the OCT system 1500, such that the algorithms and image enhancement methods can be implemented on the OCT DSP 1504 or optionally on a second dedicated DSP compensation device (COMP OCT) 1510.

The compensation parameters (n, TE, compensation method) can be determined by the application running on the computer 1506 and either used directly in the OCT DSP 1504 or transferred to the optional COMP OCT 1510, via respective feedback loops 1512, 1514, to select the compensation algorithm and/or fine tune the processing. As will be appreciated by a person skilled in the art, with the current technology, the addition of the dedicated processing unit for compensation, if needed, represents a small additional cost given the actual capacity of the DSP devices and the limited amount of calculations required for running the compensation algorithms in example embodiments. The optional polar transform unit 1509, which can be parameterized from the user-application running on the computer 1506, may also be integrated in the optional COMP OCT 1510, rather than being implemented by a dedicated DSP or processing unit, or the polar transform may be performed by the computer 1506 before the display 1508.

In one embodiment, an optical coherence tomography system comprises a detector for detecting an optical coherence tomography signals from a sample; a processing unit for converting the detected signals to raw data; a compensation unit for performing compensation to generate compensated data; and a display for displaying at least the compensated data.

The system may further comprise a feedback loop for adjustment of acquisition and/or compensation parameters for non-deferred processing. The feedback loop may be configured for extracting a functional based on multiple compensations performed on raw data from the sample, said functional depending on selected compensation parameters to determine an optimum value for at least one of the selected compensation parameters. The feedback loop may be configured for extracting a functional for respective different compensation algorithms to determine an optimum compensation algorithm.

The compensation unit may be integrated with the processing unit or is provided as a dedicated unit. The compensation unit may be configured for real time generation of the compensated data or is configured for deferred generation of the compensated data.

The compensation unit may be configured to perform a compensation algorithm using

$$\{ \begin{matrix}{I}_{i , j}^{SC} = \frac{{I}_{i , j}^{n}}{2. {M}_{i , j}} & ( a ) \\ {M}_{i , j} = {\sum }_{k = i}^{N} {I}_{k , j}^{n} & ( b )\end{matrix} \qquad Equation 1$$

to produce compensated intensities I_{i,j}^{SC}, where M_{i,j} is the compensation profile (1/M_{i,j} being the amplification factor) for each A-scan j and A-scan pixel at depth i (i=0: top of the image; i=N: bottom of the image); and n is an exponent that controls contrast; wherein Equation 1-a may be used without the coefficient 2.

The compensation unit may be configured to perform a compensation algorithm using

$$\{ \begin{matrix}{E}_{i , j} = {\sum }_{k = i}^{N} {[ {I}_{k , j}^{n} ]}^{2} & ( a ) \\ {E}_{l}^{max} = {max}_{j} {E}_{i , j} & ( b ) \\ {i}^{stop} = 1 \text{/} {E}_{l}^{max} < {10}^{- TE} . {E}_{0}^{max} & ( c ) \\ {M}_{i , j} = \{ \begin{matrix}{\sum }_{k = i}^{N} {I}_{k , j}^{n} & o < {i}_{stop} \\ {\sum }_{k = {i}_{stop}}^{N} & {I}_{k , j}^{n} i \geq {i}_{stop}\end{matrix} & ( d ) \\ {I}_{i , j}^{AC} = {I}_{i , j}^{n} \text{/} 2. {M}_{i , j} & ( e )\end{matrix} \qquad Equation 2$$

to estimate the maximum penetration depth i^{stop} (Equation 2-c) using the global maximum energy profile of all A-scans (Equation 2-b), compute the standard compensation profile I_{i,j}^{AC} for each A-scan down to the estimated depth i^{stop} (Equation 2-d and -e), maintain the compensation coefficient M_{i,j} for each A-scan, wherein M_{i,j} is the compensation profile (1/M_{i,j} being the amplification factor) for each A-scan j and A-scan pixel at depth i (i=0: top of the image; i=N: bottom of the image); n is an exponent that controls contrast; and TE is energy threshold exponent; wherein Equation 2-e may be used without the coefficient 2.

The compensation unit may be configured to perform a compensation algorithm using

$$\{ \begin{matrix}{E}_{i , j} = {\sum }_{k = i}^{N} {[ {I}_{k , j}^{n} ]}^{2} & ( a ) \\ {i}_{j}^{stop} = i \text{/} {E}_{i , j} < {10}^{- TE} . {E}_{0 , j} & ( b ) \\ {M}_{ij} = \{ \begin{matrix}{\sum }_{k = i}^{N} {I}_{k , j}^{n} & i < {i}_{j}^{stop} \\ {\sum }_{k = {i}_{j}^{stop}}^{N} & i \geq {i}_{j}^{stop}\end{matrix} & ( c ) \\ {I}_{i , j}^{DAC} = {I}_{i , j}^{n} \text{/} 2. {M}_{i , j} & ( d )\end{matrix} \qquad Equation 3$$

to compute individual A-scan maximum penetration depths iᵢ^{stop} (Equation 3-b), regularize the compensation profile M_{i,j} (Equation 3-c); generate I_{i,j}^{DAC} wherein M_{i,j} is the compensation profile (1/M_{i,j} being the amplification factor) for each A-scan j and A-scan pixel at depth i (i=0: top of the image; i=N: bottom of the image); n is an exponent that controls contrast; and TE is energy threshold exponent; wherein Equation 3-d may be used without the coefficient 2.

The compensation unit may be configured for generating the compensated data by processing the raw data. The system may be configured such that the raw data is processed by the compensation unit without prior compression/decompression processing.

The system may be configured for matching the compensated data with other diagnostic modalities and to use results of the matching to classify one or more regions of interests according to tissue characteristics. The compensation unit may be configured for using compensation in combination with other processes, for example for identifying particular tissues or structures, or for using the compensation on different sources of signals other than the detected optical coherence tomography signals.

The detection unit may be configured for detecting catheter acquired optical coherence tomography data, and the compensation unit is configured for generating the compensated data by processing the catheter acquired optical coherence tomography data. The detection unit may be configured for detecting cardiovascular optical coherence tomography pullback data, and the compensation unit is configured for generating the compensated data from the cardiovascular optical coherence tomography pullback data. The detection unit may be configured for detecting the raw data from vascular vessels or any kind of tubing or graft, and the compensation unit may be configured for generating the compensated data from the raw data from the vascular vessels or any kind of tubing or graft, for example to diagnose presence of arterial diseases. The compensation unit may be configured for enhancing particular morphological features in the arterial vessel wall or the any kind of tubing or graft being imaged.

In one embodiment, shown in FIG. 16, an optical coherence tomography image processing method 1600 comprises detecting raw optical coherence tomography data from an optical coherence tomography imaging system, 1602; applying of a compensation step for generating compensated data based on the raw data, 1604; and displaying at least the compensated data, 1606.

The method may further comprise providing a feedback loop for adjustment of acquisition and/or compensation parameters for non-deferred processing. The feedback loop may be configured for extracting a functional based on multiple compensations performed on raw data from the sample, said functional depending on selected compensation parameters to determine an optimum value for at least one of the selected compensation parameters. The feedback loop may be configured for extracting a functional for respective different compensation algorithms to determine an optimum compensation algorithm.

The compensation step may be configured for real time generation of the compensated data or is configured for deferred generation of the compensated data.

The compensation step may be configured to perform a compensation algorithm using

$$\{ \begin{matrix}{I}_{i , j}^{SC} = \frac{{I}_{i , j}^{n}}{2. {M}_{i , j}} & ( a ) \\ {M}_{i , j} = {\sum }_{k = i}^{N} {I}_{k , j}^{n} & ( b )\end{matrix} \qquad Equation 1$$

to produce compensated intensities I_{i,j}^{SC}, where M_{i,j} is the compensation profile (1/M_{i,j} being the amplification factor) for each A-scan j and A-scan pixel at depth i (i=0: top of the image; i=N: bottom of the image); and n is an exponent that controls contrast; wherein Equation 1-a may be used without the coefficient 2.

The compensation step may be configured to perform a compensation algorithm using

$$\{ \begin{matrix}{E}_{i , j} = {\sum }_{k = i}^{N} {[ {I}_{k , j}^{n} ]}^{2} & ( a ) \\ {E}_{l}^{max} = {max}_{j} {E}_{i , j} & ( b ) \\ {i}^{stop} = 1 \text{/} {E}_{l}^{max} < {10}^{- TE} . {E}_{0}^{max} & ( c ) \\ {M}_{i , j} = \{ \begin{matrix}{\sum }_{k = i}^{N} {I}_{k , j}^{n} & o < {i}_{stop} \\ {\sum }_{k = {i}_{stop}}^{N} & {I}_{k , j}^{n} i \geq {i}_{stop}\end{matrix} & ( d ) \\ {I}_{i , j}^{AC} = {I}_{i , j}^{n} \text{/} 2. {M}_{i , j} & ( e )\end{matrix} \qquad Equation 2$$

to estimate the maximum penetration depth i^{stop} (Equation 2-c) using the global maximum energy profile of all A-scans (Equation 2-b), compute the standard compensation profile I_{i,j}^{AC} for each A-scan down to the estimated depth i^{stop} (Equation 2-d and -e), maintain the compensation coefficient M_{i,j} for each A-scan, wherein M_{i,j} is the compensation profile (1/M_{i,j} being the amplification factor) for each A-scan j and A-scan pixel at depth i (i=0: top of the image; i=N: bottom of the image); n is an exponent that controls contrast; and TE is energy threshold exponent; wherein Equation 2-e may be used without the coefficient 2.

The compensation step may be configured to perform a compensation algorithm using

$$\{ \begin{matrix}{E}_{i , j} = {\sum }_{k = i}^{N} {[ {I}_{k , j}^{n} ]}^{2} & ( a ) \\ {i}_{j}^{stop} = i \text{/} {E}_{i , j} < {10}^{- TE} . {E}_{0 , j} & ( b ) \\ {M}_{ij} = \{ \begin{matrix}{\sum }_{k = i}^{N} {I}_{k , j}^{n} & i < {i}_{j}^{stop} \\ {\sum }_{k = {i}_{j}^{stop}}^{N} & i \geq {i}_{j}^{stop}\end{matrix} & ( c ) \\ {I}_{i , j}^{DAC} = {I}_{i , j}^{n} \text{/} 2. {M}_{i , j} & ( d )\end{matrix} \qquad Equation 3$$

to compute individual A-scan maximum penetration depths iᵢ^{stop} (Equation 3-b), regularize the compensation profile Mᵢ (Equation 3-c); generate i_{i,j}^{DAC} wherein M_{i,j} is the compensation profile (1/M_{i,j} being the amplification factor) for each A-scan j and A-scan pixel at depth i (i=0: top of the image; i=N: bottom of the image); n is an exponent that controls contrast; and TE is energy threshold exponent; wherein Equation 3-d may be used without the coefficient 2.

The compensation step may be configured for generating the compensated data by processing the raw data. The raw data may be processed by the compensation step without prior compression/decompression processing.

The method may further comprise matching the compensated data with other diagnostic modalities and to use results of the matching to classify one or more regions of interests according to tissue characteristics. The compensation step may be configured for using compensation in combination with other processes, for example for identifying particular tissues or structures, or for using the compensation on different sources of signals other than the detected optical coherence tomography signals.

The detecting may comprise detecting catheter acquired optical coherence tomography data, and the compensation step is configured for generating the compensated data by processing the catheter acquired optical coherence tomography data. The detecting may comprise detecting cardiovascular optical coherence tomography pullback data, and the compensation step is configured for generating the compensated data from the cardiovascular optical coherence tomography pullback data. The detecting may comprise detecting the raw data from vascular vessels or any kind of tubing or graft, and the compensation step may be configured for generating the compensated data from the raw data from the vascular vessels or any kind of tubing or graft, for example to diagnose presence of arterial diseases. The compensation step may be configured for enhancing particular morphological features in the arterial vessel wall or the any kind of tubing or graft being imaged.

It will be appreciated by a person skilled in the art that numerous variations and/or modifications may be made to the present invention as shown in the specific embodiments without departing from the spirit or scope of the invention as broadly described. The present embodiments are, therefore, to be considered in all respects to be illustrative and not restrictive. Also, the invention includes any combination of features, in particular any combination of features in the patent claims, even if the feature or combination of features is not explicitly specified in the patent claims or the present embodiments.

1. An optical coherence tomography system comprising:
a detector for detecting an optical coherence tomography signals from a sample;
a processing unit for converting the detected signals to raw data;
a compensation unit for performing compensation to generate compensated data; and
a display for displaying at least the compensated data.

2. The system of claim 1, further comprising a feedback loop for adjustment of acquisition and/or compensation parameters for non-deferred processing.

3. The system of claim 2, wherein the feedback loop is configured for extracting a functional based on multiple compensations performed on raw data from the sample, said functional depending on selected compensation parameters to determine an optimum value for at least one of the selected compensation parameters.

4. The system of claim 3, wherein the feedback loop is configured for extracting a functional for respective different compensation algorithms to determine an optimum compensation algorithm.

5. The system of claim 1, wherein the compensation unit is integrated with the processing unit or is provided as a dedicated unit and/or wherein the compensation unit is configured for real time generation of the compensated data or is configured for deferred generation of the compensated data.

6. (canceled)

7. The system of claim 1, wherein the compensation unit is configured to perform a compensation algorithm using
$$\{ \begin{matrix}{I}_{i , j}^{SC} = \frac{{I}_{i , j}^{n}}{2. {M}_{i , j}} & ( a ) \\ {M}_{i , j} = {\sum }_{k = i}^{N} {I}_{k , j}^{n} & ( b )\end{matrix} \qquad Equation 1$$
to produce compensated intensities I_{i,j}^{SC}, where M_{i,j} is the compensation profile (1/M_{i,j} being the amplification factor) for each A-scan j and A-scan pixel at depth i (i=0: top of the image; i=N: bottom of the image); and n is an exponent that controls contrast; wherein Equation 1-a may be used without the coefficient 2.

8. The system of claim 1, wherein the compensation unit is configured to perform a compensation algorithm using
$$\{ \begin{matrix}{E}_{i , j} = {\sum }_{k = i}^{N} {[ {I}_{k , j}^{n} ]}^{2} & ( a ) \\ {E}_{l}^{max} = {max}_{j} {E}_{i , j} & ( b ) \\ {i}^{stop} = 1 \text{/} {E}_{l}^{max} < {10}^{- TE} . {E}_{0}^{max} & ( c ) \\ {M}_{i , j} = \{ \begin{matrix}{\sum }_{k = i}^{N} {I}_{k , j}^{n} & o < {i}_{stop} \\ {\sum }_{k = {i}_{stop}}^{N} & {I}_{k , j}^{n} i \geq {i}_{stop}\end{matrix} & ( d ) \\ {I}_{i , j}^{AC} = {I}_{i , j}^{n} \text{/} 2. {M}_{i , j} & ( e )\end{matrix} \qquad Equation 2$$
to estimate the maximum penetration depth i^{stop} (Equation 2-c) using the global maximum energy profile of all A-scans (Equation 2-b), compute the standard compensation profile I_{i,j}^{AC} for each A-scan down to the estimated depth i^{stop} (Equation 2-d and -e), maintain the compensation coefficient M_{i,j} for each A-scan, wherein M_{i,j} is the compensation profile (1/M_{i,j} being the amplification factor) for each A-scan j and A-scan pixel at depth i (i=0: top of the image; i=N: bottom of the image); n is an exponent that controls contrast; and TE is energy threshold exponent; wherein Equation 2-e may be used without the coefficient 2.

9. The system of claim 1, wherein the compensation unit is configured to perform a compensation algorithm using
$$\{ \begin{matrix}{E}_{i , j} = {\sum }_{k = i}^{N} {[ {I}_{k , j}^{n} ]}^{2} & ( a ) \\ {i}_{j}^{stop} = i \text{/} {E}_{i , j} < {10}^{- TE} . {E}_{0 , j} & ( b ) \\ {M}_{ij} = \{ \begin{matrix}{\sum }_{k = i}^{N} {I}_{k , j}^{n} & i < {i}_{j}^{stop} \\ {\sum }_{k = {i}_{j}^{stop}}^{N} & i \geq {i}_{j}^{stop}\end{matrix} & ( c ) \\ {I}_{i , j}^{DAC} = {I}_{i , j}^{n} \text{/} 2. {M}_{i , j} & ( d )\end{matrix} \qquad Equation 3$$
to compute individual A-scan maximum penetration depths iⱼ^{stop} (Equation 3-b), regularize the compensation profile M_{i,j} (Equation 3-c); generate I_{i,j}^{DAC} wherein M_{i,j} is the compensation profile (1/M_{i,j} being the amplification factor) for each A-scan j and A-scan pixel at depth i (i=0: top of the image; i=N: bottom of the image); n is an exponent that controls contrast; and TE is energy threshold exponent; wherein Equation 3-d may be used without the coefficient 2.

10. The system of claim 1, wherein the compensation unit is configured for generating the compensated data by processing the raw data and/or wherein the system is configured such that the raw data is processed by the compensation unit without prior compression/decompression processing.

11. (canceled)

12. The system of claim 1, wherein the system is configured for matching the compensated data with other diagnostic modalities and to use results of the matching to classify one or more regions of interests according to tissue characteristics, and/or wherein the compensation unit is configured for using compensation in combination with other processes, or for using the compensation on different sources of signals other than the detected optical coherence tomography signals and/or wherein the detection unit is configured for detecting, catheter acquired optical coherence tomography data, and the compensation unit is configured for generating the compensated data by processing the catheter acquired optical coherence tomography data and/or wherein the detection unit is configured for detecting cardiovascular optical coherence tomography pullback data, and the compensation unit is configured for generating the compensated data from the cardiovascular optical coherence tomography pullback data, and/or wherein the detection unit is configured for detecting the raw data from vascular vessels or any kind of tubing or graft, and the compensation unit is configured for generating the compensated data from the raw data from the vascular vessels or any kind of tubing or graft and/or wherein the compensation unit is configured for enhancing particular morphological features in the arterial vessel wall or the any kind of tubing or graft being imaged.

13. (canceled)

15. (canceled)

16. (canceled)

17. (canceled)

18. (canceled)

19. An optical coherence tomography image processing method comprising:
detecting raw optical coherence tomography data from an optical coherence tomography imaging system;
applying of a compensation step for generating compensated data based on the raw data; and
displaying at least the compensated data.

20. The method of claim 19, further comprising providing a feedback loop for adjustment of acquisition and/or compensation parameters for non-deferred processing.

21. The system of claim 20, wherein the feedback loop is configured for extracting a functional based on multiple compensations performed on raw data from the sample, said functional depending on selected compensation parameters to determine an optimum value for at least one of the selected compensation parameters.

22. The system of claim 21, wherein the feedback loop is configured for extracting a functional for respective different compensation algorithms to determine an optimum compensation algorithm.

23. The method of claim 19, wherein the compensation step is configured for real time generation of the compensated data or is configured for deferred generation of the compensated data.

24. The method of claim 19, wherein the compensation step is configured to perform a compensation algorithm using
$$\{ \begin{matrix}{I}_{i , j}^{SC} = \frac{{I}_{i , j}^{n}}{2. {M}_{i , j}} & ( a ) \\ {M}_{i , j} = {\sum }_{k = i}^{N} {I}_{k , j}^{n} & ( b )\end{matrix} \qquad Equation 1$$
to produce compensated intensities I_{i,j}^{SC}, where M_{i,j} is the compensation profile (1/M_{i,j} being the amplification factor) for each A-scan j and A-scan pixel at depth i (i=0: top of the image; i=N: bottom of the image); and n is an exponent that controls contrast; wherein Equation 1-a may be used without the coefficient 2.

25. The method of claim 19, wherein the compensation step is configured to perform a compensation algorithm using
$$\{ \begin{matrix}{E}_{i , j} = {\sum }_{k = i}^{N} {[ {I}_{k , j}^{n} ]}^{2} & ( a ) \\ {E}_{l}^{max} = {max}_{j} {E}_{i , j} & ( b ) \\ {i}^{stop} = 1 \text{/} {E}_{l}^{max} < {10}^{- TE} . {E}_{0}^{max} & ( c ) \\ {M}_{i , j} = \{ \begin{matrix}{\sum }_{k = i}^{N} {I}_{k , j}^{n} & o < {i}_{stop} \\ {\sum }_{k = {i}_{stop}}^{N} & {I}_{k , j}^{n} i \geq {i}_{stop}\end{matrix} & ( d ) \\ {I}_{i , j}^{AC} = {I}_{i , j}^{n} \text{/} 2. {M}_{i , j} & ( e )\end{matrix} \qquad Equation 2$$
to estimate the maximum penetration depth i^{stop} (Equation 2-c) using the global maximum energy profile of all A-scans (Equation 2-b), compute the standard compensation profile I_{i,j}^{AC} for each A-scan down to the estimated depth i^{stop} (Equation 2-d and -e), maintain the compensation coefficient M_{i,j} for each A-scan, wherein M_{i,j} is the compensation profile (1/M_{i,j} being the amplification factor) for each A-scan j and A-scan pixel at depth i (i=0: top of the image; i=N: bottom of the image); n is an exponent that controls contrast; and TE is energy threshold exponent; wherein Equation 2-e may be used without the coefficient 2.

26. The method of claim 19, wherein the compensation step is configured to perform a compensation algorithm using
$$\{ \begin{matrix}{E}_{i , j} = {\sum }_{k = i}^{N} {[ {I}_{k , j}^{n} ]}^{2} & ( a ) \\ {i}_{j}^{stop} = i \text{/} {E}_{i , j} < {10}^{- TE} . {E}_{0 , j} & ( b ) \\ {M}_{ij} = \{ \begin{matrix}{\sum }_{k = i}^{N} {I}_{k , j}^{n} & i < {i}_{j}^{stop} \\ {\sum }_{k = {i}_{j}^{stop}}^{N} & i \geq {i}_{j}^{stop}\end{matrix} & ( c ) \\ {I}_{i , j}^{DAC} = {I}_{i , j}^{n} \text{/} 2. {M}_{i , j} & ( d )\end{matrix} \qquad Equation 3$$
to compute individual A-scan maximum penetration depths iⱼ^{stop} (Equation 3-b), regularize the compensation profile M_{i,j} (Equation 3-c); generate I_{i,j}^{DAC} wherein M_{i,j} is the compensation profile (1/M_{i,j} being the amplification factor) for each A-scan j and A-scan pixel at depth i (i=0: top of the image; i=N: bottom of the image); n is an exponent that controls contrast; and TE is energy threshold exponent; wherein Equation 3-d may be used without the coefficient 2.

27. The method of claim 19, wherein the compensation step is configured for generating the compensated data by processing the raw data and/or the raw data is processed by the compensation step without prior compression/decompression processing and/or further comprising matching the compensated data with other diagnostic modalities and to use results of the matching to classify one or more regions of interests according to tissue characteristics and/or wherein the compensation step is configured for using compensation in combination with other processes, or for using the compensation on different sources of signals other than the detected optical coherence tomography signals and/or wherein the detecting comprises detecting catheter acquired optical coherence tomography data, and the compensation step is configured for generating the compensated data by processing the catheter acquired optical coherence tomography data and/or wherein the detecting comprises detecting cardiovascular optical coherence tomography pullback data, and the compensation step is configured for generating the compensated data from the cardiovascular optical coherence tomography pullback data and/or wherein the detecting comprises detecting the raw data from vascular vessels or any kind of tubing or graft, and the compensation step is configured for generating the compensated data from the raw data from the vascular vessels or any kind of tubing or graft and/or wherein the compensation step is configured for enhancing particular morphological features in the arterial vessel wall or the any kind of tubing or graft being imaged.

28.

29.

30. (canceled)

31. (canceled)

32. (canceled)

33. (canceled)

34. (canceled)
